(19) United States
(12) Patent Application Publication
(10) Pub. No.: US 2016/0287423 A1
(43) Pub. Date: Oct. 6, 2016

(54) ARTIFICIAL KNEE JOINT

(71) Applicant: FILLAUER EUROPE AB, Sollentuna (SE)

(72) Inventor: Christoffer Ramirez, Vallentuna (SE)

(21) Appl. No.: 15/037,202

(22) PCT Filed: Dec. 8, 2014
(86) PCT No.: PCT/SE2014/051467

§ 371 Date: May 17, 2016

(30) Foreign Application Priority Data
Dec. 19, 2013 (SE) 1351537-4

(51) Int. Cl. A61F 5/01

(52) U.S. Cl. CPC A61F 5/0123; A61F 2002/503

(57) ABSTRACT

There is shown an artificial joint (1), which joint has a first part (11) adapted to be attached at a wearer's first body part or substitute body part, and a second part (12) adapted to be attached to a second body part or substitute body part, wherein the first part (11) and the second part (12) can be rotated relatively each other and are connected to each other by means of at least four shafts (21, 22, 23, 24) and corresponding links (13, 14, 15). The joint (1) is characterised in that it is freely moveable when the joint is unloaded by the wearer, and in loaded position the same joint is geometrically locked.

### TECHNICAL FIELD

The present invention relates to an artificial knee joint provided as a mechanical joint construction adapted for stabilising a lower extremity and supporting a normal walking step of the user.

### BACKGROUND

Walking is a complicated process and to be able to work efficiently, the lower extremities have to be able to 1: carry the body weight during the supporting phase, 2: rotate and coordinate the joints to provide a progression in the walking direction, 3: adjust the length of the extremity by means of bending the knee joint during the swing-phase, and 4: further decrease the movement of the body weight in height direction by means of bending the knee joint slightly during midstance (phase in the middle of the step). Lack of any of these functions can adventure a person's ability to walk.

Nevertheless, hinder of movement can be lessened by means of conventional orthosis-support by means of for instance a KAFO (Knee Ankle Foot Orthosis). A KAFO has to, to be energy-efficient, give full stability in the supporting phase and a natural and non-hindered swing-phase, as well as admit some bending of the knee joint in the middle of the step without adventuring the knee stability. An artificial knee joint also has to be designed such that it follows the natural poly-centric/multi-shaft pattern of movement of the knee.

For stabilisation of the human knee joint of individuals having reduced muscle strength and/or reduced joint control, to a large extent, knee joints having a locking function are used for providing the user stability during walking. These joints are only unlocked to provide a sitting position with bent knee. This locking has the disadvantage that the users will have an unnatural pattern of walking being energy-inefficient and in the long-run will give arise to wear damages.

Artificial knee joints by which the knee joint can be unlocked when initiating the swing phase are known within the technical field. U.S. Pat. No. 5,490,831, U.S. Pat. No. 2,943,622 and the European patent application EP0872224 disclose knee joints using a pressure against the heel to provide a locking signal. According to these publications, a detector element is provided in the heel, which detecting element is connected to a locking element, for instance by means of a cable or rod. As long as the detector element detects a pressure against the heel, the joint is locked by means of the locking element. As long as there is no pressure against the heel, the knee joint is then released.

A significant disadvantage with the locking according to said document is that the knee joint also will be released if pressure is exerted against the frontal part of the user's foot. If the user tries to overcome a hindrance, such as a kerb, using his leg, the detector element will not detect any pressure against the heel and will hence not release the articulated joint. The user's knee joint will hence fold. As a result of this limitation the solution is unpractical and also dangerous.

Patent U.S. Pat. No. 4,632,096 describes a joint that can be released by means of the foot being twisted relatively the leg. A cable is attached to the foot, which cable is connected to a locking element in the knee joint. By twisting the foot the locking of the joint can be released by means of the cable.

The disadvantage with the solution according to this US patent is that the release only can be performed by means of actively twisting the foot. This implies that the user has to be ready to twist his foot in each step. Furthermore, according to this solution, is also required a link between the knee joint and the foot or foot joint. This implies the functionality of the system depending on the structure of the surface of the ground.

Within the prosthesis technology, wherein user has no knee joint at all following an amputation, there are the so-called four-shaft knee joints for providing stability in the design of the knee joint. One example is given in patent U.S. Pat. No. 5,181,931. Typical for these solutions is that the technology requires that the user does not have any own lower leg, and these solutions are thus disqualified as aiding means for many of the users of which the present invention is related to.

### SUMMARY OF THE INVENTION

The present invention solves the problems with prior art by means of the features of the appended claims. The invention also provides a solution that does not require any external mechanical control by means of wire connections or electronic control system to provide stability in the supporting phase. The geometrical stability of the knee joint according to the invention is also by its spring-tensioned sliding shaft adjustable for a wide range of weight classes.

According to a preferred embodiment of the present invention there is provided an artificial knee joint, which has a first part adapted to be attached at a wearer's first body part or a substitute body part and a second part adapted to be attached at a second body part or substitute body part, which knee joint comprises a first part in which an upper frontal shaft and an upper rear shaft are mounted, a to the first part rotatably connected second part in which a lower frontal shaft and a lower rear shaft are mounted, a frontal link rotatably mounted on the frontal shafts,

a rear link rotatably mounted on the rear shafts,
a stop provided on the first part and provided for limiting the rotary motion of the frontal link around the upper frontal shaft. The lower rear shaft is slidably mounted in a groove formed in the second part, which in relation to the lower frontal shaft extends between a frontal and a distal end, wherein the shaft is adjustably pre-tensioned in a direction of the distal end.

Few parts and simple mechanics render the invention unique in its simplicity and reliability, wherein other solutions strive to be complex and hence too sensitive.

The polycentric movement of the joint of the present invention imitates the movement of the anatomic knee in a natural way such that the user is not limited in his range of movement.

### BRIEF DESCRIPTION OF DRAWINGS

Embodiments of the present invention will be explained in more detail with reference to appended drawing figures, wherein:

FIG. 1 illustrates a view of a knee joint according to the invention incorporated in an artificial knee-ankle-foot orthosis (KAFO),

FIG. 2 illustrates the knee joint in mounted position,

FIG. 3 illustrates the specific components of the knee joint,

FIG. 4 illustrates a geometric model of the knee joint,

FIG. 5 illustrates the position of the knee joint over the thibio-femural joint, in sagittal view, and

FIG. 6-14 illustrate the function of the joint seen in relation to the different phases of the walking cycle.

### DETAILED DESCRIPTION OF PREFERRED EMBODIMENTS

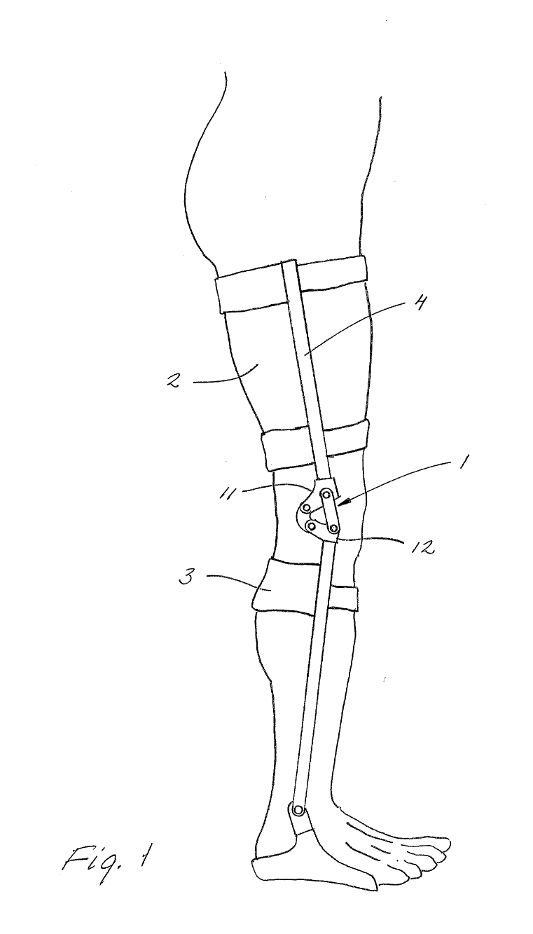

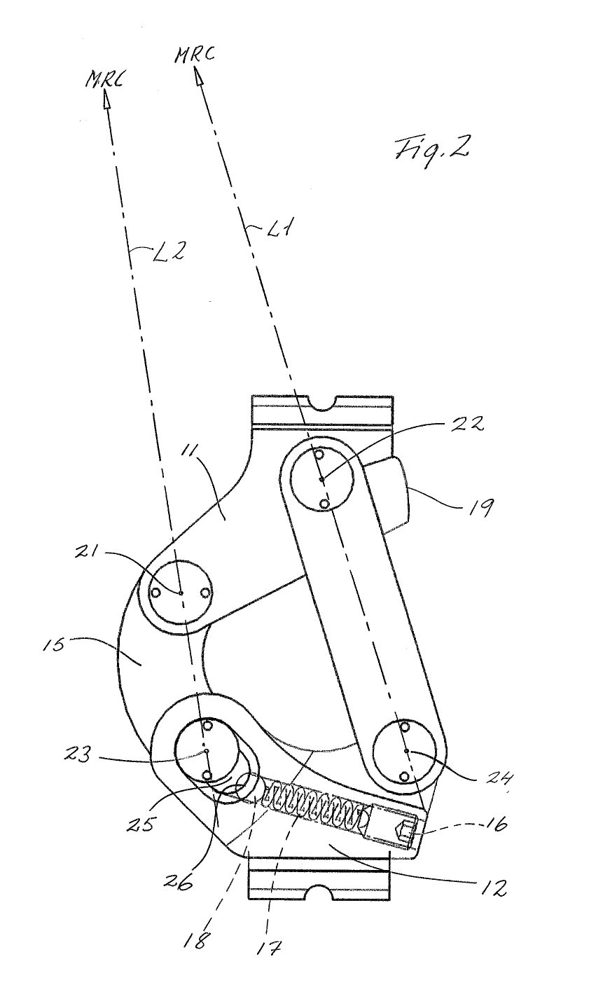

FIG. 1 illustrates the invention embodied as an artificial knee joint 1 being incorporated in an orthopaedic means of assistance. The knee joint 1 comprise a first part 11 being rotatably connected to a second part 12 by means of links 13 and 14 as well as the link 15, such as illustrated in FIG. 2.

The knee joint 1 is attachable to a user's leg 2. The first part 11 is adapted to be connected to a separately manufactured segment 4 attachable to the thigh of the wearer. Part 12 is in a similar way connectable to another separately manufactured segment 4 being attachable to the lower leg of the wearer.

FIG. 2 illustrates the knee joint in its initial position and can, depending on the (power)torques acting through the joint, either be released for rotation or locked by means of the links part 15 and 13-14 being positioned in parallel. This is further explained below with reference to FIGS. 6-15.

FIG. 3 in more detail illustrates the individual components of the invention. Part 11 is the upper part of the joint being attachable to the locking segment of the orthopaedic means of assistance, and part 12 is attachable to the segment of the lower leg. The links being illustrated by part 13, 14 and 15 connect part 11 and part 12 by means of the shaft being illustrated by the parts 21-24. Part 19 is a stop for the links 13 and 14 such that they do not move too long in their movement.

Part 18 is a ball being slidably supporting on a lower end of part 15, and together with a spring 17 and an adjustment screw 16 provides an adjustable adjustment of the force that has to be overcome for the shaft 23 to move downwards/forwards in a in the part 12 formed recess, in which the shaft 23 is movably mounted. In this way, the geometric stability of the knee joint can be adapted to different wearers. The adjustment screw 16 with the spring 17 and the ball 18 is typically inserted and mounted into a boring with internal threads of the lower part 12 of the knee joint and provided for this purpose.

A preferred embodiment of the orthopaedic knee joint thus comprises a first part 11, in which an upper frontal shaft 22 and an upper rear shaft 21 are mounted. In a to the first part 11 articulately connected second part 12, a lower frontal shaft 24 and a lower rear shaft 23 are mounted. A frontal link 13, 14 is rotatably mounted on the frontal shafts 22 and 24, and a rear link 15 is rotatably mounted on the rear shafts 21, 23. A stop 19 is provided on the first part 11 and provided for limiting the rotational motion of the frontal link 13, 14 around the upper frontal shaft 22. The lower rear shaft 23 is slidably mounted in a groove 25 formed in the second part 12, which in relation to the lower frontal shaft 24 extends between a frontal 26 and a distal end 27, wherein the shaft 23 is adjustably pre-tensioned 16, 17, 18 in a direction of the distal end 27.

The pre-tension of the lower rear shaft 23 is generated by means of an against the shaft 23 supporting ball 18 which is actuated by a in the second part 12 mounted spring 17 having a corresponding adjustment screw 16.

The frontal shafts 24 and 22 are on a frontal line L1 and the rear shafts 21, 23 are on a rear line L2, which at extension of the two lines intersects the frontal line L1 at an angle, in an intersectional point MRC in the loaded and straight position of the knee joint is outside the knee joint. The groove 25 has an extension in the second part 12, such that a movement of the shaft 23 in the frontal end 26 of the groove decreases said angle, and moves the intersectional point MRC of the lines further away from the knee joint.

To be more precise, the angle between the frontal line L1 and the rear line L2 will be zero or close to zero when the shaft 23 positions in an end position in the frontal end 26 of the groove 25.

The centre distance between the two shafts 22, 24 is larger than the centre distance between the rear shafts 21, 23, whereby the relation between the length of the frontal link 13, 14 and the length of the rear link 15 results in that the intersectional point MRC is positioned between the frontal shaft 22, 24 in the unloaded, maximum bent position of the knee joint.

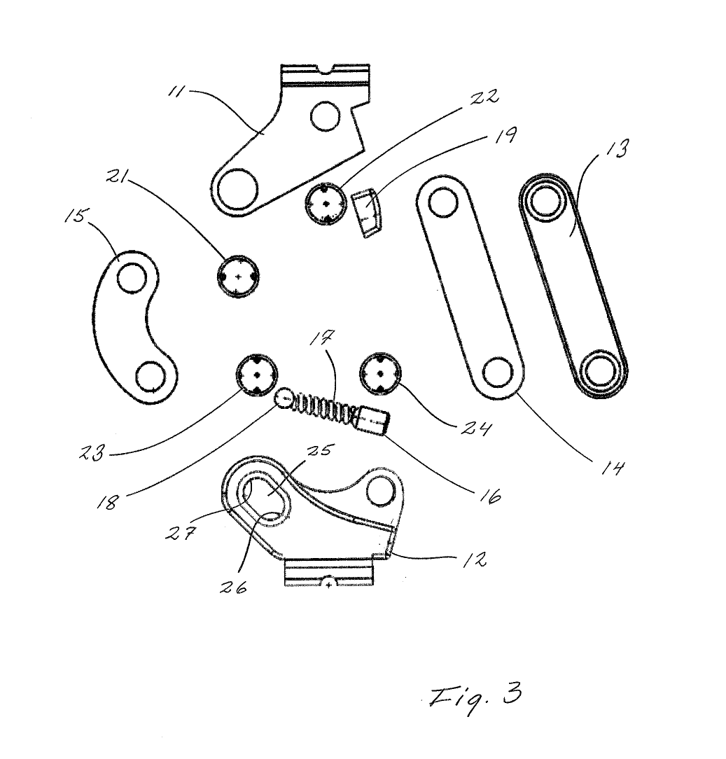

FIG. 4 illustrates the theoretical model of the present invention to be able to provide a basic stability by means of a so-called increased instantaneous rotational centre, hereinafter referred to as MRC. This model's function suits the users well that normally require orthosis-support by means of a KAFO, i. e. persons having reduced muscle strength from the hip and below in the extremity. The knee joint 1 has a short rear link between shaft 21 and 23 and a set of longer links between shaft 22 and 24. The intersectional point, i. e. the instantaneous rotational centre MRC between these two links, will be positioned behind the wearer's hip joint (in the extension of the arrows L1-L2 in FIG. 2). Since MRC is initially projected behind the hip joint, there is provided a joint being hyper stable throughout the supporting phase, and since it is almost in the same height as the hip joint, there is provided a joint that can be initiated to bending at the end of the supporting phase by means of the wearer generating a small bending torque. The anatomic knee joint is not a monocentric joint but a polycentric joint such that it is more suitable to the movement pattern of this mechanical type of joint together with the anatomic part.

The positioning of the invention on the knee of the wearer must be performed carefully to provide the very best functioning.

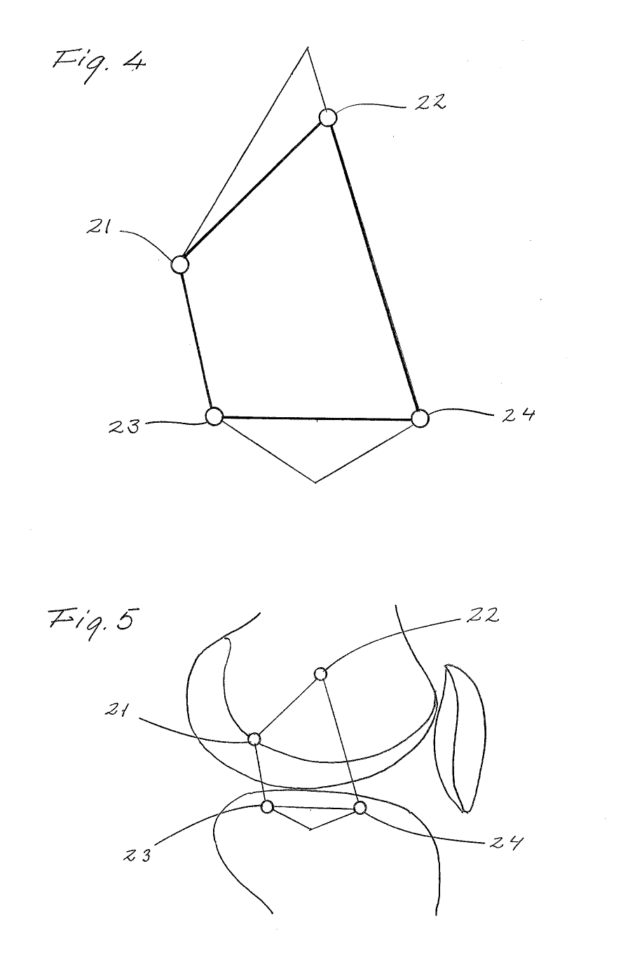

FIG. 5 illustrates where, in relation to the anatomic knee joint, the invention has to be positioned.

By letting shaft 23 to slide downwards in said groove formed in the part 12 there is provided a slight bending of the knee joint of about 5-10 degrees, as well as link 15 is given the possibility to position parallel to the links 13 and 14. This moves MRC infinitely far away out such that the joint will be stable to that extent that it is not possible to initiate a bending of the knee joint as longs as this geometric locking is present.

FIG. 6. Initial Contact: Start of the walking cycle. When the floor reaction force (GRK) is directed anterior if MRC, a stretching torque is generated in the joint which locks the movement of the knee joint, but since GRK is moved closer to shaft 24, an energy saving bending is activated in the knee joint, provided by the bending component, and also providing the joint to be geometrically stable.

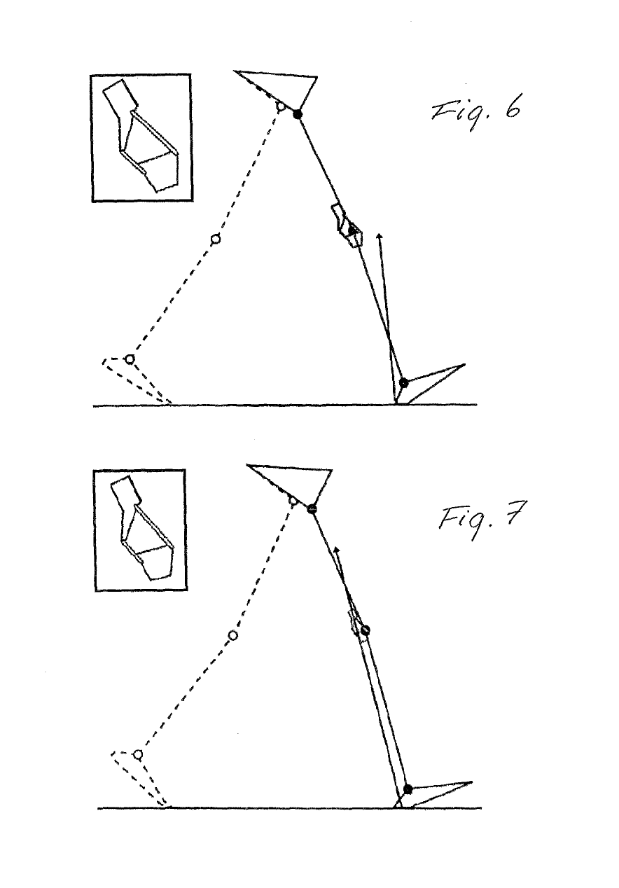

FIG. 7. Loading Response: In this position during the walking cycle the links of the joint have become totally parallel and the joint is completely stable.

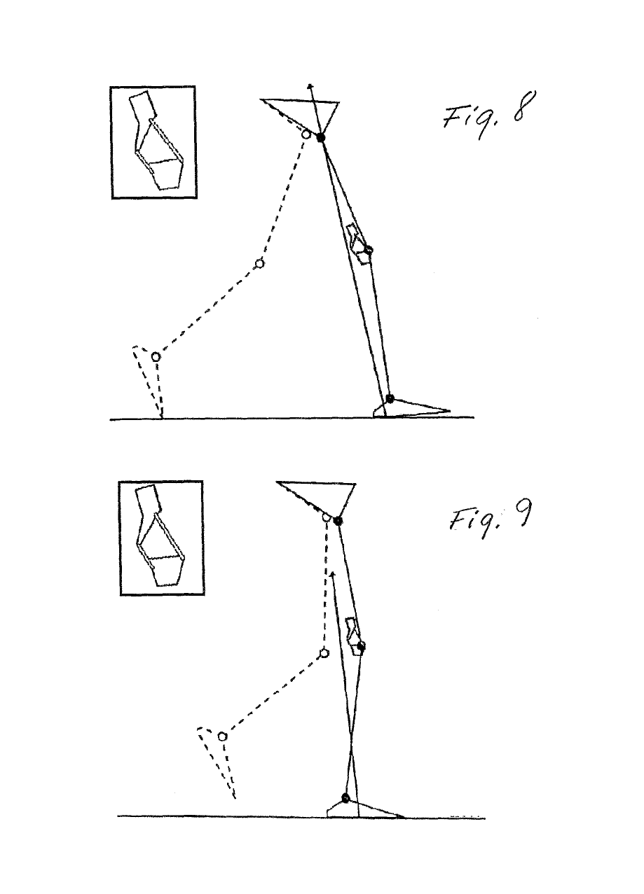

FIG. 8. Opposite Toe Off: When the supporting leg supports the total load of the body and the bending component is in maximum position, the knee joint has 7 degrees bending to decrease the vertical movement of the weight of the body. This provides a more energy efficient walking, which is important in particular for users having reduced muscle strength.

FIG. 9. Midstance: Mainly the same condition as in previous part of the walking cycle. GRK has such a direction that the knee of the wearer could not be stable without normal muscle strength, unless the geometric stability of the present invention providing this.

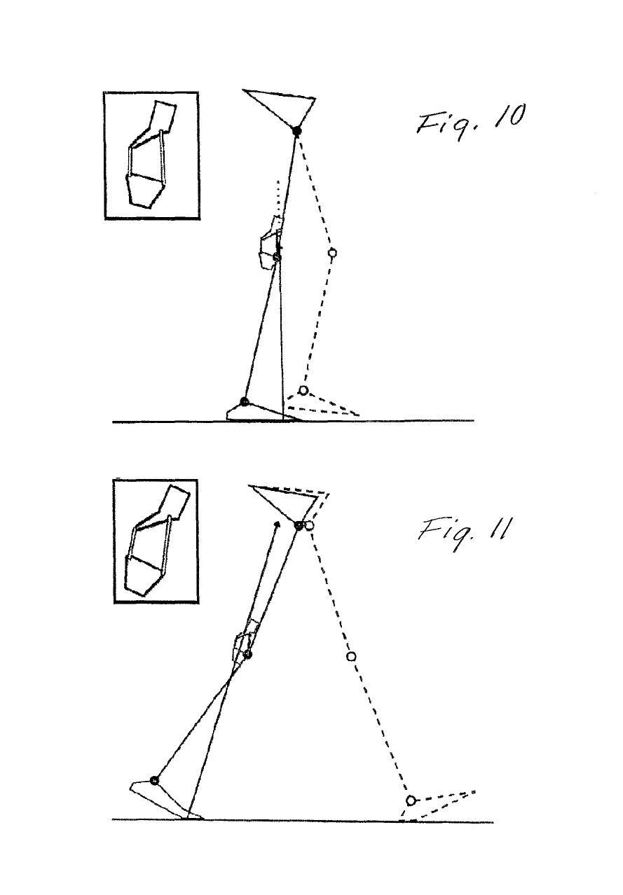

FIG. 10. Heel Rise: When the distance at right angles of GRK to shaft 24 is decreased, the bending component will be deactivated gradually to provide the switching to swing phase.

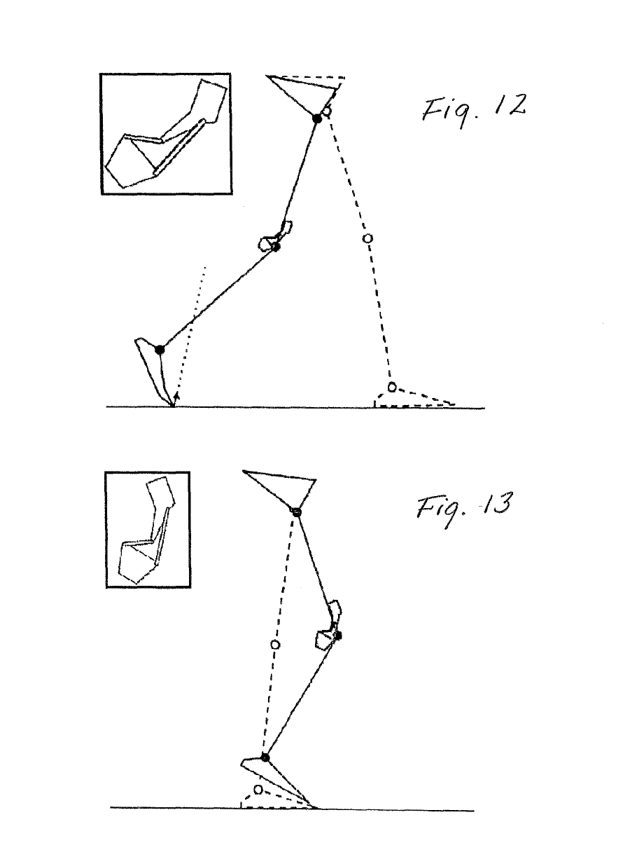

FIG. 11. Opposite Initial Contact: Having the bending component deactivated, the joint will be fully straight, and stable as long as GRK is directed anterior to the MRC of the joint. The wearer can in this position also control if the joint should be bent by generating a bending torque in the hip. This manually moves GRK posterior to MRC and in that way a swing phase is initiated. Usually this process occurs at ca 55% of the length of the walking cycle.

FIG. 12. Toe Off: Following Opposite Initial Contact the supporting phase rapidly approach its end and the wearer can either initiate a swing phase by generating a bending torque in the hip joint, or wait until GRK in a natural way moves posterior to MRC, or simply by lifting the leg from the ground. By bending the knee joint, MRC will be moved quickly to the same level as the knee joint 1. Already at 13 degrees bending in the anatomical knee MRC has moved downwards to shaft 22 of the knee joint 1.

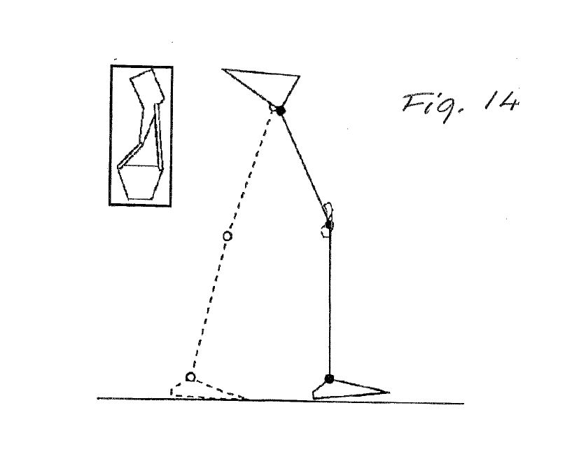

FIG. 13. Feet Adjacent: In the swing phase the knee joint of the present invention rotates around a MRC positioned on the level of the joint, for angles over 13 degrees, and moves with the anatomic joint centre. The maximum knee bending angle the wearer has in the swing phase is related to the walking speed as well as to the hip strength the wearer has. Lower walking speed gives a lower maximum bending of the knee joint in the swings phase, and lower muscle strength in the hip bending also gives lower bending of the knee joint in the swing phase.

FIG. 14. Tibia Vertical: Now the swing phase is approaching its end and a new walking cycle starts with Initial Contact. Between Tibia Vertical and Initial Contact the joint goes against full extension.

This invention discloses a theoretical and practical model for a geometric stand phase-stability incorporated in an orthosis joint provided for supporting a remaining extremity or substitute for a shortened extremity, as well as gives a user the opportunity to an energy efficient walking with a simple and adaptive knee joint orthosis.

As described above there is provided an artificial knee joint which has a first part adapted to be attached to a wearer's first body part or substitute body part and a second part adapted to be attached to a second body part or substitute body part, wherein the first part and the second part can be rotated in relation to each other and are connected to each other via at least four shafts and corresponding links. Characterising for the mechanical knee joint according to the invention is that it is freely movable when the joint is unloaded by the wearer, but in loaded position it is geometrically locked. In particular one of the shafts is slidably mounted in such a way that the geometry of the system of links can be changed such that the joint is locked. Another characterising feature is that the loading condition through the joint influences the stability without the assistance of further control mechanisms, and the degree of stability can be adjusted based on the needs of the wearer.

1. An artificial knee joint, having a first part adapted to be attached to a wearer's first body part or substitute body part and a second part adapted to be attached to a second body part or substitute body part, which knee joint comprises
a first part in which an upper frontal shaft and an upper rear shaft are mounted,
a to the first part articulately connected second part in which a lower frontal shaft and a lower rear shaft are mounted,
a frontal link rotatably mounted on the frontal shafts,
a rear link rotatably mounted on the rear shafts,
a stop provided on the first part and provided for limiting the rotational motion of the frontal link around the upper frontal shaft,
wherein the lower rear shaft is slidably mounted in a groove formed in the second part, which in relation to the lower frontal shaft extends between a frontal and a distal end, wherein the shaft is adjustably pre-tensioned in a direction of the distal end.

2. The artificial knee joint according to claim 1, wherein the pre-tension of the lower rear shaft is generated by means of an against the shaft supporting ball which is actuated by a in the second part mounted spring having a corresponding adjustment screw.

3. The artificial knee joint according to claim 1, wherein the frontal shaft are on a frontal line, and the rear shaft are on a rear line, which at the extension of the two lines intersects the frontal line at an angle, in an intersectional point in the loaded and straight position of the knee joint is outside the knee joint, wherein the groove has an extension in the second part, such that a movement of the shaft in a direction directed towards the frontal end of the groove decreases said angle, and moves the intersectional point of the lines further away from the knee joint.

4. The artificial knee joint according to claim 3, wherein the angle between the frontal line and the rear line is zero or close to zero when the shaft positions in an end position in the frontal end of the groove.

5. The artificial knee joint according to claim 3, wherein the centre distance between the two shafts is larger than the centre distance between the rear shafts, whereby the relation between the length of the frontal link and the length of the rear link results in that the intersectional point is positioned between the frontal shafts in the unloaded, maximum bent position of the knee joint.
